(19) United States
(12) Patent Application Publication
(10) Pub. No.: US 2021/0062177 A1
(43) Pub. Date: Mar. 4, 2021

(54) Alginate Lyase

(71) Applicant: Ocean University of China, Qingdao (CN)

(72) Inventors: Xiangzhao Mao, Qingdao (CN); Zhen Liu, Qingdao (CN); Danyang Cheng, Qingdao (CN); Changhu Xue, Qingdao (CN)

(73) Assignee: Ocean University of China, Qingdao (CN)

(21) Appl. No.: 16/989,258

(22) Filed: Aug. 10, 2020

(30) Foreign Application Priority Data
Sep. 3, 2019 (CN) 2019108292848

(51) Int. Cl. C12N 9/88; C12N 15/63

(52) U.S. Cl. CPC C12N 9/88; C12N 15/63

(57) ABSTRACT

The disclosure provides a novel alginate lyase, whose amino acid sequence is SEQ ID NO: 1. The disclosure further provides a nucleic acid encoding the alginate lyase, which can have the sequence of SEQ ID NO: 2, and may be comprised in a recombinant expression vector. Such an expression vector may be comprised in a host microorganism to express the alginate lyase. Also disclosed are methods of producing the alginate lyase by expression in a host microorganism. The alginate lyase enzyme provided by the disclosure can be used for catalyzing the degradation of sodium alginate to produce unsaturated 2-8 saccharides, among which unsaturated disaccharides and unsaturated trisaccharides can be the main component.

### TECHNICAL FIELD

The invention belongs to the technical field of functional enzyme screening, in particular to a novel PL-6 family alginate lyase ScCD6, its enzymatic properties, and products.

### BACKGROUND

Brown algae is one of the three major algaes, and its main components are alginate, mannitol and laminarin. Among them, the related researches on the utilization of mannitol and laminarin are relatively mature. Therefore, how to efficiently degrade alginate and realize its industrial applications are currently hot research topics. Alginate is a linear polysaccharide composed of β-D-mannuronic acid (M) and α-L-guluronic acid (G). Its degradation product, alginate oligosaccharide, has important physiological activities, such as antioxidant, anti-tumor and promoting plant root cell growth, so it has attracted extensive attentions.

Alginate lyase (Aly) can degrade alginate through the β-elimination reaction, and form unsaturated carbon-carbon double bonds at the non-reducing ends of the degradation products, thus generating a special absorption peak at 235 nm in the ultraviolet band that can be used for product detection. According to the substrate specificity of Aly, it can be divided into poly M, poly G and bifunctional alginate lyase. According to its enzyme digestion methods, it can be divided into endo-type and exo-type alginate lyases. The end product of endo-type Aly degrading alginate is alginate oligosaccharides (AOs), and the polymerization degree of the common product is from 2 to 6. The end product of exo-type Aly is monosaccharide in most cases, but there are also reports that the disaccharide is the smallest digestion unit of some Aly. According to the classification of CAZY database, alginate lyase belongs to the polysaccharide lyase (PL) and is specifically divided into seven families: PL-5, PL-6, PL-7, PL-14, PL-15, PL-17 and PL-18.

At present, the preparation methods of alginate oligosaccharides are mainly chemical methods, such as acidolysis, which have severe preparation conditions and are easy to cause environmental pollutions. In contrast, the degradation conditions of alginate polysaccharides by biological methods are mild, easy to control and environmentally friendly. Therefore, it is necessary to develop highly efficient, stable, and easily prepared alginate lyases.

### SUMMARY

The invention provides a novel PL-6 alginate lyase ScCD6, and uses the enzyme to degrade sodium alginate to produce alginate oligosaccharide.
Firstly, the invention provides an alginate lyase, whose amino acid sequence SEQ ID NO: 1 is:

MNRPRLSTYAAITVAAITIAALTTAASALASTGDTSSRTPSGNSTPQANA
TIVNVSSSTQLTTAMANAVAGQTIVLANGSYSIGKLNAKNGTSSAPITIM
AAQQGKAIITGGQLEVLSSSYVTFSGLKWTNSNTLKITSSHHIRLTRNHF
RLTESSSLKWIIIQGANSHHNRIDHNLFEEKHQLGNFITIDGSSTQQSQY
DLIDYNHFRNIGPRATNEMEAIRVGWSAISKSDGFTTVENNLFENCDGDP
EIVSVKSNANTVRYNTFRTSQGSVSLRHGNRSQVHGNFFFGGGKTGTGGV
RVYGQDHKIYNNHFEGLTGTGYDAALQLDGGDVDTSGALSSHWRVYRATA
VHNTFVNNVSNIEIGANYSLAPVDSLVADNIVVGSSGKLFNELKMPKNMT
YAGNIGWPTGSATIGITTGVRTVNPLLAKQGEVYRLGTGSPAVNTASGSY
SFLADDMDGQSRSGTADVGADELSTGTVVHKPLNSADVGISAP.

One specific nucleotide sequence of the gene encoding the above enzyme is SEQ ID NO: 2, and its sequence is as follows:

ATGAACAGACCAAGGCTCAGCACCTACGCCGCGATCACCGTCGCGGCCAT
CACGATCGCGGCCCTCACCACCGCAGCATCCGCTCTCGCCTCCACCGGCG
ATACGTCGTCGCGCACCCCGAGCGGGAACTCGACCCCACAGGCCAACGCC
ACCATCGTCAACGTCTCGTCGTCGACGCAGTTGACCACCGCGATGGCCAA
CGCCGTAGCCGGCCAAACGATCGTCCTCGCCAACGGCTCCTACTCGATCG
GCAAGCTCAACGCCAAGAACGGCACCTCCAGCGCACCCATCACGATCATG
GCCGCCCAACAGGGCAAGGCGATCATCACCGGAGGGCAGCTCGAGGTTCT
CAGCTCGTCGTACGTGACGTTCTCCGGGCTGAAGTGGACGAACAGCAACA
CGTTGAAGATCACCAGTTCGCACCACATCCGGTTAACCCGCAACCACTTC
CGGCTCACCGAGTCGAGCTCGTTGAAGTGGATCATCATCCAGGGAGCCAA
CAGCCACCACAACCGGATCGACCACAACCTGTTCGAGGAGAAGCACCAGC
TCGGCAACTTCATCACCATCGACGGGTCTTCGACCCAGCAGTCGCAGTAC
GACCTGATCGACTACAACCACTTTCGCAACATCGGTCCGCGCGCCACCAA
CGAGATGGAGGCGATCCGGGTCGGCTGGAGTGCGATCTCCAAGTCGGACG
GGTTCACCACGGTCGAGAACAACCTCTTCGAGAACTGCGACGGCGACCCT
GAGATCGTCTCCGTGAAGAGCAACGCCAACACCGTCCGGTACAACACCTT
CCGGACATCACAGGGCTCGGTGTCCCTGCGCCACGGCAACCGCAGCCAGG
TCCACGGCAACTTCTTCTTCGGAGGCGGCAAGACCGGCACCGGCGGCGTC
CGGGTCTACGGCCAGGACCACAAGATATACAACAACCACTTCGAAGGACT
GACCGGCACCGGCTACGACGCGGCGCTGCAACTCGACGGCGGGGACGTCG
ACACCTCAGGCGCGCTCTCCTCGCACTGGCGGGTGTACCGGGCCACGGCA
GTGCACAACACCTTCGTCAACAACGTCTCGAACATCGAGATCGGCGCCAA
CTACAGTCTCGCCCCGGTCGACAGCCTGGTCGCCGACAACATCGTCGTCG
GTTCTTCCGGCAAGCTCTTCAACGAGCTGAAGATGCCCAAGAACATGACG
TACGCGGGCAACATCGGCTGGCCGACGGGTTCCGCCACCATCGGGATCAC
CACCGGCGTCCGCACCGTGAACCCGCTCCTGGCCAAGCAGGGTGAGGTCT
ACCGCCTCGGCACCGGTAGCCCCGCGGTGAACACCGCGTCGGGTAGCTAC
AGCTTCCTCGCCGATGACATGGACGGTCAGTCACGCAGTGGAACGGCCGA
TGTCGGAGCGGACGAACTGTCCACCGGCACCGTGGTCCACAAGCCGCTCA
ACTCCGCCGACGTCGGAATCAGCGCCCCCTGA.

Secondly, the invention provides a recombinant expression vector, which carries a gene encoding the above-mentioned alginate lyase.

The invention also provides a recombinant host for recombinant expression of the alginate lyase.

The alginate lyase in the invention can degrade sodium alginates into unsaturated oligosaccharides with polymerization degree of 2-8, among which the unsaturated disaccharides is dominant since it accounts for about 70% of the total unsaturated oligosaccharide products. The enzyme can also degrade poly M and poly G polysaccharide fragments and produce corresponding M oligosaccharides and G oligosaccharides, respectively.

Further, an optimum temperature of the reaction is 50° C. and an optimum pH is 9.0.

Further, the concentration of substrate (sodium alginate, poly M, poly G) in the reaction can be 0.3% (w/v). An amount of added enzyme can be 0.6 U. A reaction can be carried out at 30° C., pH 7.0 for 12 hours. The obtained products can be used for HPLC tests.

Further, the enzyme has a good stability under the conditions of 40° C. and pH 7.0-10.0. 80% of the enzyme activity can be retained at 30° C. for 24 hours, and 80% of the enzyme activity can be retained at 4° C. for 4 days at pH 7.0-10.0.

Further, the enzyme can be used to catalyze sodium alginate to produce unsaturated 2-8 saccharides, and the main product is unsaturated disaccharides, which can be prepared to obtain unsaturated disaccharides and unsaturated trisaccharides.

### BRIEF DESCRIPTION OF THE DRAWINGS

FIGS. 1A-1C: Diagrams of amino acid sequence analysis (FIG. 1A), phylogenetic tree analysis (FIG. 1B) and multi-sequence alignment analysis (FIG. 1C) of alginate lyase ScCD6 in the invention (SEQ ID NOS: 1, 3-7).

FIG. 2: SDS-PAGE diagrams of purify alginate lyase ScCD6 in the invention, wherein M is a broad-spectrum protein Marker (11-180 kDa); 1 is the supernatant of fermentation broth; 2 is the supernatant penetrate liquid of the fermentation broth; 3 is the 40 mM imidazole eluent; 4 is the 80 mM imidazole eluent; 5 is the cell breakage fluid; 6 is the cell breakage fluid penetrating fluid; 7 is the 40 mM imidazole eluent; 8 is the 80 mM imidazole eluent.

FIGS. 3A-3F: Enzymatic property diagrams of the alginate lyase ScCD6 in the invention, wherein FIG. 3A is the optimum reaction temperature; FIG. 3B is the optimum reaction pH; FIG. 3C is the temperature stability; FIG. 3D is the pH stability; FIG. 3E is the effects of metal ions and chemical reagents on ScCD6 enzyme activity; FIG. 3F is the substrate specificity.

FIGS. 4A-4C: Product analysis diagram of polysaccharide degradation by alginate lyase ScCD6 in the invention, wherein FIG. 4A is the result diagram of sodium alginate degradation; FIG. 4B is the result diagram of poly M degradation; FIG. 4C is the result diagram of poly G degradation.

### DETAILED DESCRIPTION

The present invention uses conventional techniques and methods used in the field of molecular biology and enzyme catalysis, which are described in detail below with reference to examples and attached figures.

As used herein, the term “protein” refers broadly to a polymer of two or more amino acids joined together by peptide bonds. The term “protein” also includes molecules which contain more than one protein joined by a disulfide bond, or complexes of proteins that are joined together, covalently or noncovalently, as multimers (e.g., dimers, tetramers). Thus, the terms peptide, oligopeptide, and polypeptide are all included within the definition of protein and these terms are used interchangeably.

A protein may be “structurally similar” to a reference protein if the amino acid sequence of the protein possesses a specified amount of sequence similarity and/or sequence identity compared to the reference protein. Thus, a protein may be “structurally similar” to a reference protein if, compared to the reference protein, it possesses a sufficient level of amino acid sequence identity, amino acid sequence similarity, or a combination thereof.

The present invention includes variants of SEQ ID NO: 1 and SEQ ID NO: 2. A variant of the protein of SEQ ID NO: 1 may include one or more substitution, deletion, and/or insertion mutations relative to the sequence of SEQ ID NO: 1. Thus, the terms variant enzyme,” variant protein,” “variant polypeptide,” “modified amino acid sequence” or “modified polypeptide,” which can be used interchangeably, refer to an amino acid sequence that is different from the reference polypeptide by one or more amino acids, e.g., by one or more amino acid substitutions, deletions, and/or additions. For example, a variant protein may include 1, 2, 3, 4, 5, 6, 7, 8, 9, 10, or more substitution, deletion, and/or insertion mutations.

In an aspect, a variant is a “functional variant” which retains some or all of the functions of the reference polypeptide. The term “functional variant” further includes conservatively substituted variants. The term “conservatively substituted variant” refers to a peptide having an amino acid sequence that differs from a reference peptide by one or more conservative amino acid substitutions, and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

As used herein, the term “polynucleotide” refers to a polymeric form of nucleotides of any length, either ribonucleotides, deoxynucleotides, peptide nucleic acids, or a combination thereof, and includes both single-stranded molecules and double-stranded duplexes. A polynucleotide can be obtained directly from a natural source, or can be prepared with the aid of recombinant, enzymatic, or chemical techniques. In one embodiment, a polynucleotide is isolated. A polynucleotide can be linear or circular in topology. A polynucleotide can be, for example, a portion of a vector, such as an expression or cloning vector, or a fragment. Variant polynucleotides include polynucleotides encoding variant proteins.

The invention includes polynucleotides that are complementary to SEQ ID NO: 2, complementary to the complement of SEQ ID NO: 2, and/or complementary to a polynucleotide encoding SEQ ID NO: 1, and substantial complements thereof. The terms “complement” and “complementary” as used herein, refer to the ability of two single stranded polynucleotides to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary. The term “substantial complement” and cognates thereof as used herein, refer to a polynucleotide that is capable of selectively hybridizing to a specified polynucleotide under stringent hybridization conditions. Stringent hybridization can take place under a number of pH, salt and temperature conditions. The pH can vary from 6 to 9, preferably 6.8 to 8.5. The salt concentration can vary from 0.15 M sodium to 0.9 M sodium, and other cations can be used as long as the ionic strength is equivalent to that specified for sodium. The temperature of the hybridization reaction can vary from 30° C. to 80° C., preferably from 45° C. to 70° C. Additionally, other compounds can be added to a hybridization reaction to promote specific hybridization at lower temperatures, such as at or approaching room temperature. Among the compounds contemplated for lowering the temperature requirements is formamide. Thus, a polynucleotide is typically substantially complementary to a second polynucleotide if hybridization occurs between the polynucleotide and the second polynucleotide. As used herein, “specific hybridization” refers to hybridization between two polynucleotides under stringent hybridization conditions.

As used herein, an “isolated” substance is one that has been removed from a cell and many of the proteins, nucleic acids, and other cellular material of its natural environment are no longer present. A substance may be purified, i.e., at least 60% free, at least 75% free, or at least 90%, 95%, or 99% free from other components with which they are naturally associated. Proteins and polynucleotides that are produced by recombinant, enzymatic, or chemical techniques are considered to be isolated by definition, since they are produced free from other components with which they might be naturally associated.

As used herein, the terms “coding region,” “coding sequence,” and “open reading frame” are used interchangeably and refer to a nucleotide sequence that encodes a protein and, when placed under the control of appropriate regulatory sequences expresses the encoded protein. The boundaries of a coding region are generally determined by a translation start codon at its 5′ end and a translation stop codon at its 3′ end.

A “regulatory sequence” is a nucleotide sequence that regulates expression of a coding sequence to which it is operably linked. Nonlimiting examples of regulatory sequences include promoters, enhancers, transcription initiation sites, translation start sites, translation stop sites, transcription terminators, and poly(A) signals. The term “operably linked” refers to a juxtaposition of components such that they are in a relationship permitting them to function in their intended manner A regulatory sequence is “operably linked” to a coding region when it is joined in such a way that expression of the coding region is achieved under conditions compatible with the regulatory sequence.

The invention provides expression vectors comprising nucleotides encoding SEQ ID NO: 1, and variations thereof, operably connected to regulatory sequences suitable for the expression of the encoded protein. The selection of a suitable vector, or the construction of a suitable vector will depend on the intended host cell and is within the ordinary skill of one in the art. For example, a pET-28a expression vector comprising SEQ ID NO:1 operably linked to a promoter is suitable for expression of ScCD6 in E. coli BL21 (DE3) cells.

Expression vectors can be exogenously introduced into suitable host cells as will be well understood by persons of ordinary skill in the art. “Exogenous” refers to a protein or polynucleotide, which is not normally or naturally found in a wild-type cell. An exogenous polynucleotide may include, in some embodiments, a coding sequence that is normally present in a wild-type cell but is operably linked to a regulatory region to which it is not normally operably linked, or vice versa. In some embodiments an exogenous polynucleotide may encode an endogenous protein. As used herein, the terms “endogenous protein” and “endogenous polynucleotide” refer to a protein or polynucleotide that is normally or naturally found in a cell microbe. An “endogenous polynucleotide” is also referred to as a “native polynucleotide.”

As used herein, “heterologous amino acid sequence” refers to amino acid sequences that are not normally present as part of a protein present in a wild-type cell. For instance, “heterologous amino acid sequence” includes extra amino acids at the amino terminal end or carboxy terminal of a protein that are not normally part of a protein.

The term “and/or” means one or all of the listed elements or a combination of any two or more of the listed elements. The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention. For any method disclosed herein that includes discrete steps, the steps may be conducted in any feasible order. And, as appropriate, any combination of two or more steps may be conducted simultaneously.

In accordance with the foregoing, disclosed herein are:

An isolated alginate lyase comprising:

a) an isolated enzyme having the amino acid sequence of SEQ ID NO: 1; or,

b) an isolated enzyme having the functions of an enzyme having the amino acid sequence of SEQ ID NO: 1 comprising one or more substitution, deletion, and/or insertion mutations relative to the sequence of SEQ ID NO: 1.

An isolated nucleic acid encoding the alginate lyase.

The isolated nucleic acid comprising the nucleotide sequence of SEQ ID NO: 2.

A recombinant expression vector comprising a nucleic acid encoding the alginate lyase.

A recombinant host microorganism comprising an exogenous recombinant expression vector comprising a nucleic acid encoding the alginate lyase.

A method of making the alginate lyase comprising expressing alginate lyase of claim in a host microorganism comprising an exogenous recombinant expression vector comprising a nucleic acid encoding the alginate lyase.

A method for preparing alginate oligosaccharides comprising degrading sodium alginate by contacting the sodium alginate with the alginate lyase to prepare alginate oligosaccharide product, wherein, in some embodiments, reaction conditions for the degrading step comprise pH 9.0 and a temperature of 50° C., and in some embodiments the concentration of sodium alginate in the reaction is 0.3%, the reaction time is 12 hours, and the amount of added enzyme is 0.6 U, where, preferably, the alginate oligosaccharide product comprises saccharides having polymerization numbers of 2-8 and a main product is unsaturated disaccharides.

### EXAMPLE 1

### Source and Sequence Analysis of the Novel Alginate Lyase

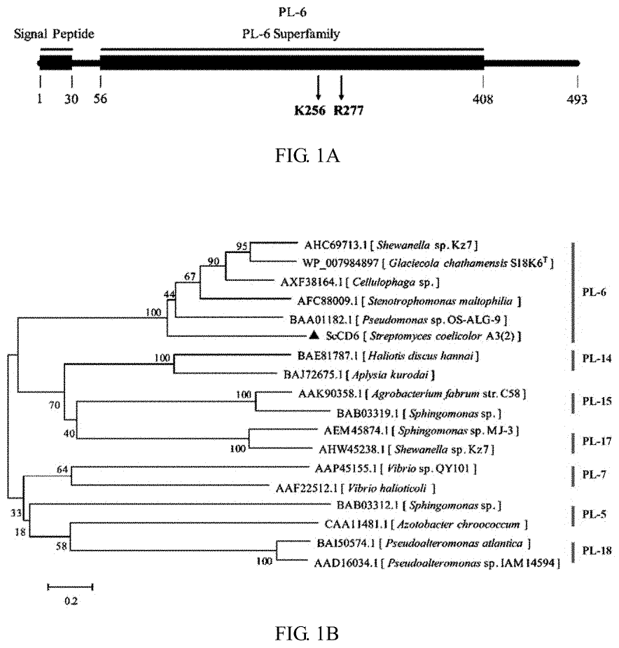

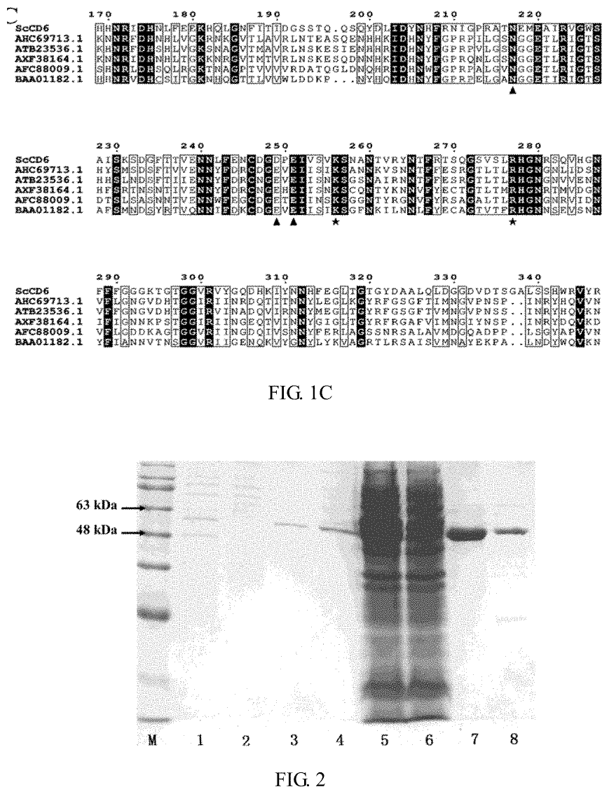

The whole genome of Streptomyces coelicolor A3(2) was sequenced to obtain the ScCD6 sequence, which was 1482 bases long and encoded a protein containing 493 amino acids. The sequence was submitted to NCBI for BLAST analysis. The analysis results predicted that the protein was a PL-6 family alginate lyase, and the SignalP 4.1 analysis results showed that the N terminal of the sequence contained a signal peptide with 30 amino acids long. MEGA6.0 was used to analyze the phylogenetic tree of alginate lyase sequences from different families. The results confirmed that the coding genes did belong to the PL-6 family. In addition, multiple sequence alignment analysis was carried out by using software ClustalX and online analysis website EScript 3.0. The analysis results showed that ScCD6 had the highest sequence similarity with the polyMG-specific alginate lyase (GenBank access no. AFC88009.1) from Stenotrophomonas maltophia, with a similarity of about 27.68% (FIGS. 1A-1C).

In order to further predict the physical and chemical properties of the enzyme, the sequence was submitted to Expasy online analysis website for analysis. The analysis results showed that the molecular mass of the enzyme was 52.12 kDa, the isoelectric point was 7.89, and the stability coefficient was 27.83, which confirmed that the enzyme could be considered as a relatively stable protein.

### EXAMPLE 2

### Heterologous Expression and Purification of Alginate Lyase

The target protein gene was recombined with pET-28a plasmid vector by seamless splicing, and Escherichia coli BL21 (DE3) was used as the expression host to obtain an engineered strain capable of producing alginate lyase ScCD6. LB culture and IPTG was used to induce the expression of the target protein. The supernatant of fermentation broth and cells were collected by centrifugation and the cells were broken to collect the crude extracts. The supernatant of the fermentation broth and the crude extracts were purified by Ni column. Imidazole eluents with different concentration gradients were used for elution. The concentration gradients of imidazole eluents used were respectively 0 mM, 10 mM, 20 mM, 40 mM, 80 mM, 100 mM, 120 mM, 200 mM and 500 mM. The imidazole eluents with different concentrations were obtained by diluting with 20 mM pH 8.0 PBS buffers (500 mM NaC1). The results showed that 40 mM and 80 mM imidazoles could elute the target protein and obtain a single band.

### EXAMPLE 3

### Enzymatic Property Study of Alginate Lyase

Using 0.3% (w/v) sodium alginate as the substrate, in a 200 μL reaction system, after reaction for 20 min and boiling 2 min for inactivating, DNS method was used to determine the content of reducing sugar in the product to characterize the enzyme activity.

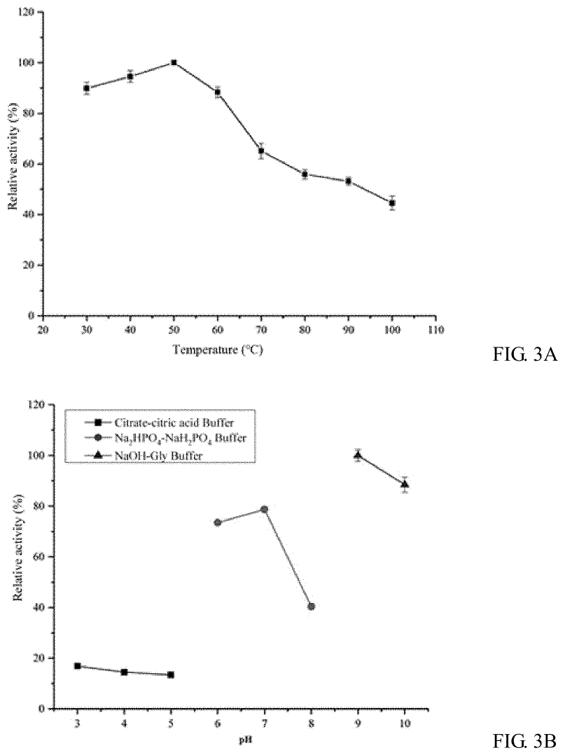

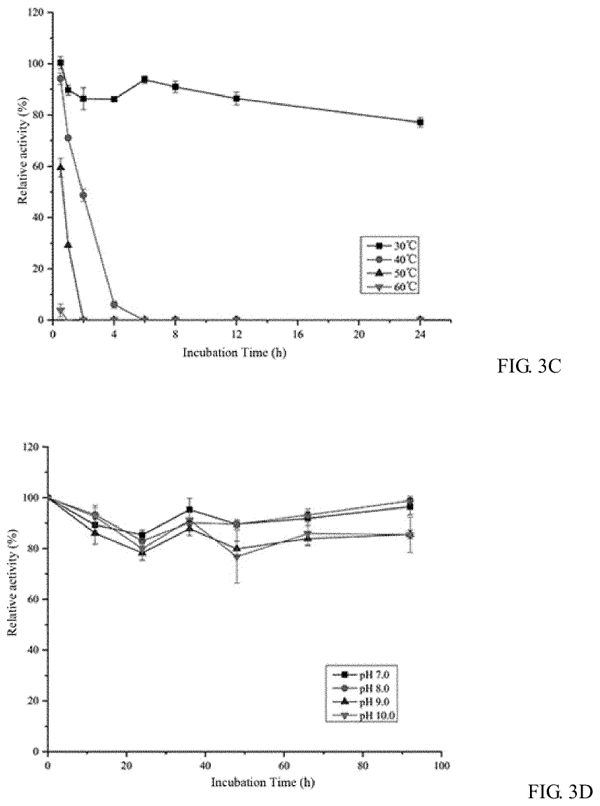

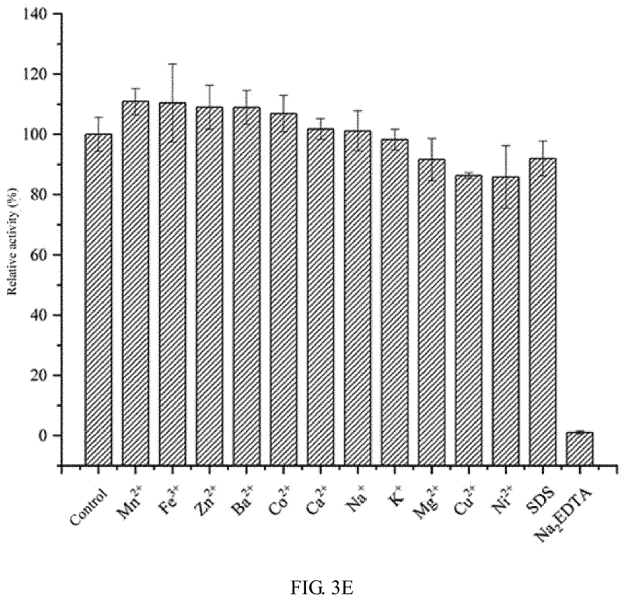

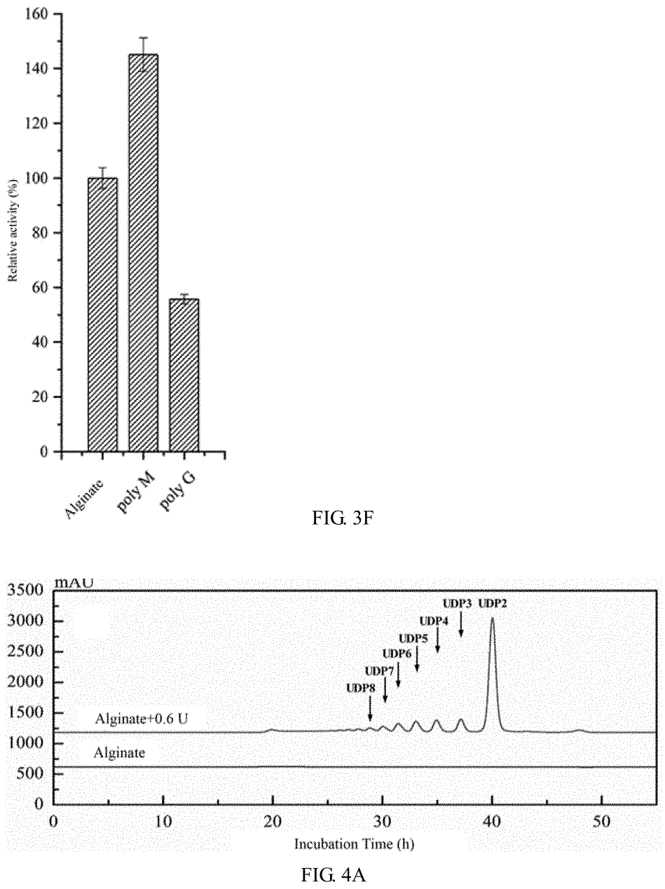

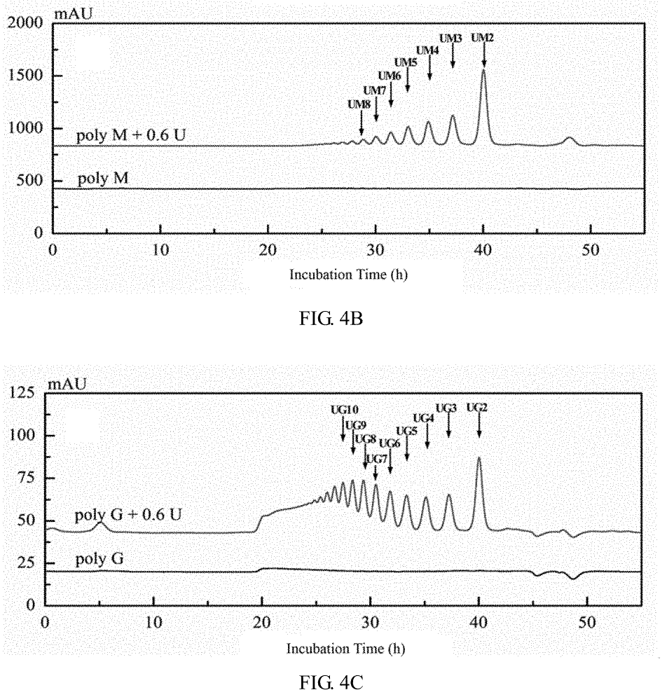

The optimum reaction temperature of ScCD6 was 50° C. under the conditions of pH 7.0 and temperature range of 30-100° C. Under the conditions of 50° C. and pH 3.0-10.0, the optimum pH is 9.0. To determine the temperature stability, ScCD6 was incubated at 30° C., 40° C., 50° C. and 60° C., and its residual enzyme activity was determined by sampling at different times. When determining the stabilities of the enzyme under different pH, the enzyme was stored in buffer solution with pH 7.0, 8.0, 9.0 and 10.0 at 4° C., and the residual enzyme activity was determined by sampling at different times. The results showed that the enzyme activity of ScCD6 was very stable under 40° C. and pH 7.0-10.0. The effects of metal ions and chemical reagents on the enzyme activities showed that Mn²⁺, Fe³⁺, Zn²⁺, Ba²⁺, Co²⁺ could promote the enzyme activity, Mg²⁺, Cu²⁺, Ni²⁺ and SDS could inhibit the enzyme activity, in addition, Ca²⁺, Na⁺, K⁺ had little effects on the enzyme activity, but Na₂EDTA could make ScCD6 lose the enzyme activity, as shown in FIGS. 3A-3F. In order to investigate the substrate specificity of ScCD6, sodium alginate, poly M and poly G were used as substrates and reacted at 40° C. for 20 min. The content of reducing sugar was determined by DNS method. The results showed that ScCD6 was a poly M specific alginate lyase.

TABLE 1
Effects of Metal Ions and Chemical
Reagents on ScCD6 Enzyme Activity

| Metal Ions/ | Relative | |
|---|---|---|
| Chemical | enzyme | Standard |
| Reagents | activity % | deviation |
| Control | 100.00 | ±5.65 |
| Mn²⁺ | 110.91 | ±4.38 |
| Fe³⁺ | 110.41 | ±12.99 |
| Zn²⁺ | 109.04 | ±7.31 |
| Ba²⁺ | 108.96 | ±5.61 |
| Co²⁺ | 106.89 | ±6.08 |
| Ca²⁺ | 101.76 | ±3.40 |
| Na⁺ | 101.15 | ±6.63 |
| K⁺ | 98.28 | ±3.38 |
| Mg²⁺ | 91.64 | ±6.99 |
| Cu²⁺ | 86.33 | ±0.94 |
| Ni²⁺ | 85.86 | ±10.36 |
| SDS | 92.02 | ±5.75 |
| Na₂EDTA | 1.03 | ±0.53 |

### EXAMPLE 4

### Product Study of Polysaccharide Degradation by Alginate Lyase ScCD6

Using 0.3% (w/v) sodium alginate, poly M and poly G substrates, in a 200 μL reaction system, 0.6 U alginate lyase ScCD6 was added and reacted at 30° C. and pH 7.0 for 12 hours. The reaction product was boiled for 10 min and centrifuged at 10,000 rpm for 10 min to remove the proteins and other impurities in the system. The supernatant was collected and passed through a 0.22 μm filter membrane for HPLC test. The HPLC conditions were as follows: Superdex™ 30 Increase 30/100 Gel column, 0.2 M NH₄HCO₃ as mobile phase, flow rate of 0.4 mL/min, room temperature (˜25° C.), loading sample volume of 100 μL, UV detection at 235 nm.

The results of HPLC showed that ScCD6 could degrade all three kinds of polysaccharides, and the main degradation product was unsaturated disaccharide and 3-8 saccharides, which indicated that ScCD6, as an endo-type alginate lyase, could degrade sodium alginate to prepare unsaturated oligosaccharides with various molecular weights.

Although the examples of the invention have been shown and described, it was understood that various changes, modifications, substitutions and variations can be made to these examples without departing from the principles and spirits of the invention for the general technicians in the field. The scope of the invention is defined by the appended claims and their equivalents.

1-6. (canceled)

7. A method for preparing alginate oligosaccharides comprising degrading sodium alginate by contacting the sodium alginate with an alginate lyase having the amino acid sequence of SEQ ID NO: 1 to prepare an alginate oligosaccharide product.

8. The method of claim 7, wherein reaction conditions for the degrading step comprise pH 9.0 and a temperature of 50° C.

9. The method of claim 7, wherein the concentration of sodium alginate in the reaction is 0.3% (w/v), the reaction time is 12 hours, and the amount of added enzyme is 0.6 U.

10. The method of claim 7, wherein the alginate oligosaccharide product comprises saccharides having polymerization numbers of 2-6 and wherein a main product is unsaturated disaccharides.

11. The method of claim 7, further comprising activating the alginate lyase with a metal ion selected from the group consisting of Mn²⁺, Fe³⁺, Zn²⁺, Ba²⁺ and Co²⁺.


SEQUENCE LISTING (7 sequences)

SEQ ID NO: 1 - protein, 493 aa, Streptomyces coelicolor
MNRPRLSTYAAITVAAITIAALTTAASALASTGDTSSRTPSGNSTPQANATIVNVSSSTQLTTAMANAVAGQTIVLANGSYSIGKLNAKNGTSSAPITIMAAQQGKAIITGGQLEVLSSSYVTFSGLKWTNSNTLKITSSHHIRLTRNHFRLTESSSLKWIIIQGANSHHNRIDHNLFEEKHQLGNFITIDGSSTQQSQYDLIDYNHFRNIGPRATNEMEAIRVGWSAISKSDGFTTVENNLFENCDGDPEIVSVKSNANTVRYNTFRTSQGSVSLRHGNRSQVHGNFFFGGGKTGTGGVRVYGQDHKIYNNHFEGLTGTGYDAALQLDGGDVDTSGALSSHWRVYRATAVHNTFVNNVSNIEIGANYSLAPVDSLVADNIVVGSSGKLFNELKMPKNMTYAGNIGWPTGSATIGITTGVRTVNPLLAKQGEVYRLGTGSPAVNTASGSYSFLADDMDGQSRSGTADVGADELSTGTVVHKPLNSADVGISAP

SEQ ID NO: 2 - DNA, 1482 bp, Streptomyces coelicolor
atgaacagaccaaggctcagcacctacgccgcgatcaccgtcgcggccatcacgatcgcggccctcaccaccgcagcatccgctctcgcctccaccggcgatacgtcgtcgcgcaccccgagcgggaactcgaccccacaggccaacgccaccatcgtcaacgtctcgtcgtcgacgcagttgaccaccgcgatggccaacgccgtagccggccaaacgatcgtcctcgccaacggctcctactcgatcggcaagctcaacgccaagaacggcacctccagcgcacccatcacgatcatggccgcccaacagggcaaggcgatcatcaccggagggcagctcgaggttctcagctcgtcgtacgtgacgttctccgggctgaagtggacgaacagcaacacgttgaagatcaccagttcgcaccacatccggttaacccgcaaccacttccggctcaccgagtcgagctcgttgaagtggatcatcatccagggagccaacagccaccacaaccggatcgaccacaacctgttcgaggagaagcaccagctcggcaacttcatcaccatcgacgggtcttcgacccagcagtcgcagtacgacctgatcgactacaaccactttcgcaacatcggtccgcgcgccaccaacgagatggaggcgatccgggtcggctggagtgcgatctccaagtcggacgggttcaccacggtcgagaacaacctcttcgagaactgcgacggcgaccctgagatcgtctccgtgaagagcaacgccaacaccgtccggtacaacaccttccggacatcacagggctcggtgtccctgcgccacggcaaccgcagccaggtccacggcaacttcttcttcggaggcggcaagaccggcaccggcggcgtccgggtctacggccaggaccacaagatatacaacaaccacttcgaaggactgaccggcaccggctacgacgcggcgctgcaactcgacggcggggacgtcgacacctcaggcgcgctctcctcgcactggcgggtgtaccgggccacggcagtgcacaacaccttcgtcaacaacgtctcgaacatcgagatcggcgccaactacagtctcgccccggtcgacagcctggtcgccgacaacatcgtcgtcggttcttccggcaagctcttcaacgagctgaagatgcccaagaacatgacgtacgcgggcaacatcggctggccgacgggttccgccaccatcgggatcaccaccggcgtccgcaccgtgaacccgctcctggccaagcagggtgaggtctaccgcctcggcaccggtagccccgcggtgaacaccgcgtcgggtagctacagcttcctcgccgatgacatggacggtcagtcacgcagtggaacggccgatgtcggagcggacgaactgtccaccggcaccgtggtccacaagccgctcaactccgccgacgtcggaatcagcgccccctga

SEQ ID NO: 3 - protein, 178 aa, Shewanella sp. Kz7
KNNRFDHNHLVGKRNKGVTLAVRLNTEASQENHHKIDHNYFGPRPILGSNGGETLRIGTSHYSMSDSFTVVENNYFDRCDGEVEIISIKSANNKVSNNTFFESRGTLTLRHGNGNLIDSNVFLGNGVDHTGGIRIINRDQTITNNYLEGLKGYRFGSGFTIMNGVPNSPINRYHQVVN

SEQ ID NO: 4 - protein, 178 aa, Thalassomonas sp.
KHNRFDHSHLVGKSNAGVTMAVRLNSKESIENHHRIDHNYFGPRPVLGSNGGETLRIGTSHHSLNDSFTIIENNYFDRCNGEVEIISNKSGSNAIRNNTFFESRGTLTLRHGNGNVVENNVFFGNGVDHTGGIRVINADQVIRNNYMEGLTGYRFGSGFTVMNGVPNSSINRYHQVKN

SEQ ID NO: 5 - protein, 178 aa, Cellulophaga sp.
KNNRIDHNHLTGKKNRGVTMTVRLNSKESQDNNHKIDHNYFGPRQNLGSNGGETLRIGTSHFSRTNSNTIVENNYFDRCNGEHEIISNKSCQNTYKNNVFYECTGTLTMRHGNRTMVDGNIFIGNNKPSTGGIRIINGEQTVINNYGIGLTGYRFRGAFVIMNGIYNSPINRYDQVKD

SEQ ID NO: 6 - protein, 178 aa, Stenotrophomonas maltophilia
SHNRLDHSQLRGKTNAGPTVVVVRDATQGLDNQHRIDHNWFGPRPALGVNGGETLRIGTSDTSLSASNNTVENNWFEGCDGETEIINSKSGGNTYRGNVFYRSAGALTLRHGNGNRVIDNVFLGDDKAGTGGVRIINGDQIVSNNYFERLAGSSNRSALAVMDGQADPPLSGYAPVVN

SEQ ID NO: 7 - protein, 175 aa, Pseudomonas sp.
HHNRVDHCSITGKNHQGTTLVVWLDDKPNYHQIDHNYFGPRPELGANGGETIRIGTSAFSMNDSYRTVQNNIFDKCDGEVEIISIKSGFNKILNNLFYECAGTVTFRHGNNSEVSNNYFIANNVTNSGGVRIIGENQKVYGNYLYKVAGRTLRSAISVMNAYEKPALNDYWQVKN